(19) United States
(12) Patent Application Publication
(10) Pub. No.: US 2014/0057161 A1
(43) Pub. Date: Feb. 27, 2014

(54) Electrolyte Additive for Rechargeable Lithium Battery and Rechargeable Lithium Battery Including Same

(71) Applicant: SAMSUNG SDI CO., LTD, Yongin-si (KR)

(72) Inventors: Dae-Sik Kim, Yongin-si (KR); Jiten Singh, Yongin-si (KR); Eun-Cheol Lee, Yongin-si (KR); Maeng-eun Lee, Yongin-si (KR); Won-Il Jung, Yongin-si (KR)

(73) Assignee: SAMSUNG SDI CO., LTD, Yongin-si (KR)

(21) Appl. No.: 13/802,536

(22) Filed: Mar. 13, 2013

(30) Foreign Application Priority Data
Aug. 24, 2012 (KR) 10-2012-0092673

(51) Int. Cl. C07D 251/50; H01M 10/052; C07D 251/54; H01M 10/0567

(52) U.S. Cl. CPC C07D 251/50; H01M 10/0567; H01M 10/052; C07D 251/54
USPC 429/188; 544/204

(57) ABSTRACT

An electrolyte additive for a rechargeable lithium battery, represented by the following Chemical Formula 1:

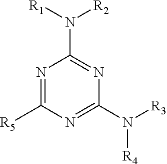

wherein, R₁ to R₄ are each independently selected from hydrogen, an aliphatic saturated hydrocarbon group, an aliphatic unsaturated hydrocarbon group, an aromatic hydrocarbon group, a heterocyclic group, or a combination thereof, provided that at least one of R₁ or R₂ and at least one of R₃ or R₄ are independently an unsaturated hydrocarbon group including at least one carbon-carbon double bond, and R₅ is selected from hydrogen, a halogen, an aliphatic saturated or unsaturated hydrocarbon group, an aromatic hydrocarbon group, a hydroxyl group, a nitro group, a cyano group, an imino group, an amino group, an amidino group, a hydrazine group, a carboxyl group, a heterocyclic group, or a combination thereof.

### RELATED APPLICATIONS

This application claims priority to and the benefit of Korean Patent Application No. 10-2012-0092673, filed in the Korean Intellectual Property Office on Aug. 24, 2012, the entire contents of which are incorporated herein by reference.

### BACKGROUND

1. Field

This disclosure relates to an electrolyte additive for a rechargeable lithium battery and a rechargeable lithium battery including the same.

2. Description of the Related Art

Since the industrial environment has changed to pursue environmentally-friendly energy, new energy sources are being researched. Particularly, a rechargeable lithium battery having a high energy density and a high performance that is also being capable of providing stable electric power is actively researched for use as a main electric energy source or an auxiliary electric energy source of an electric vehicle or a hybrid vehicle.

In view of energy density, LiCoO₂ is generally used for a positive active material for a rechargeable lithium battery, however, it has a problem in that accomplishing high voltage with it is difficult. Therefore, new materials for substituting the same are researched, and Mn-based lithium oxide has been drawing attention.

However, when a battery using 4V-grade spinel LiMn₂O₄ or 5 V-grade spinel Li₁₊ₓNi_{y}Mn_{2-y-z}M_{z}O_{4+w}, (0≦x<0.2, 0.4≦y≦0.6, 0≦z≦0.2, 0≦w≦0.1, M=Al, Ti, Mg, Zn, or a combination thereof) is allowed to stand at high temperatures in the charged state, the conventional electrolyte including a LiPF₆ lithium salt is decomposed to generate HF, and resultantly, metal ions are eluted. In addition, since the eluted metal ions are deposited on the surface of a negative electrode, the negative electrode potential is increased, and the open-circuit voltage (OCV) of the battery is dropped. Accordingly, the battery cycle performance and storage characteristics at high temperatures are deteriorated.

### SUMMARY

Aspects of embodiments of the present invention provide an electrolyte additive for a rechargeable lithium battery that prevents or reduces the elution of metal included in a positive active material at high temperatures and in addition, decreases the deposition of metal ions on the surface of a negative electrode, thereby preventing or reducing the open-circuit voltage drop that was traditionally caused when the rechargeable lithium battery was allowed to stand at high temperatures for a long time.

Another embodiment of the present invention provides an additive for an electrolyte of a rechargeable lithium battery, an electrolyte for a rechargeable lithium battery, and a rechargeable lithium battery including the electrolyte additive.

According to one embodiment of the present invention, an electrolyte additive for a rechargeable lithium battery is a compound represented by the following Chemical Formula 1:

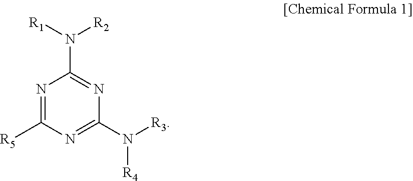

In Chemical Formula 1, R₁ to R₄ are each independently selected from hydrogen, an aliphatic saturated hydrocarbon group, an aliphatic unsaturated hydrocarbon group, an aromatic hydrocarbon group, a heterocyclic group, or a combination thereof, provided that at least one of R₁ or R₂ and at least one of R₃ or R₄ are independently an unsaturated hydrocarbon group including at least one carbon-carbon double bond. R₅ is selected from hydrogen, a halogen, an aliphatic saturated or unsaturated hydrocarbon group, an aromatic hydrocarbon group, a hydroxyl group, a nitro group, a cyano group, an imino group, an amino group, an amidino group, a hydrazine group, a carboxyl group, a heterocyclic group, or a combination thereof.

In some embodiments, in Chemical Formula 1, R₁ to R₄ are each independently selected from a C1 to C20 alkyl group, a C2 to C20 alkenyl group, a C2 to C20 alkynyl group, an allyl group, a C3 to C30 cycloalkyl group, a C3 to C30 cycloalkenyl group, a C6 to C30 aryl group, or a C2 to C30 heterocyclic group, provided that at least one of R₁ or R₂ and at least one of R₃ or R₄ are independently a C2 to C20 alkenyl group or an allyl group, and R₅ is selected from hydrogen, a halogen, C1 to C20 an alkyl group, a C2 to C20 alkenyl group, a C2 to C20 alkynyl group, an allyl group, a C3 to C30 cycloalkyl group, a C3 to C30 cycloalkenyl group, a C6 to C30 aryl group, a C2 to C30 heterocyclic group, a hydroxyl group, a nitro group, a cyano group, an imino group, an amino group, an amidino group, a hydrazine group, a carboxyl group, or a combination thereof.

In some embodiments, in Chemical Formula 1, R₁ to R₄ are each independently a vinyl group or an allyl group, R₅ is selected from a halogen, —NH₂, —NHR′, and —NR′R″, wherein R′ and R″ are each independently a C1 to C10 alkyl group.

In some embodiments, the electrolyte additive may be a compound selected from compounds of the following Chemical Formulae 1a to 1d, or a mixture thereof:

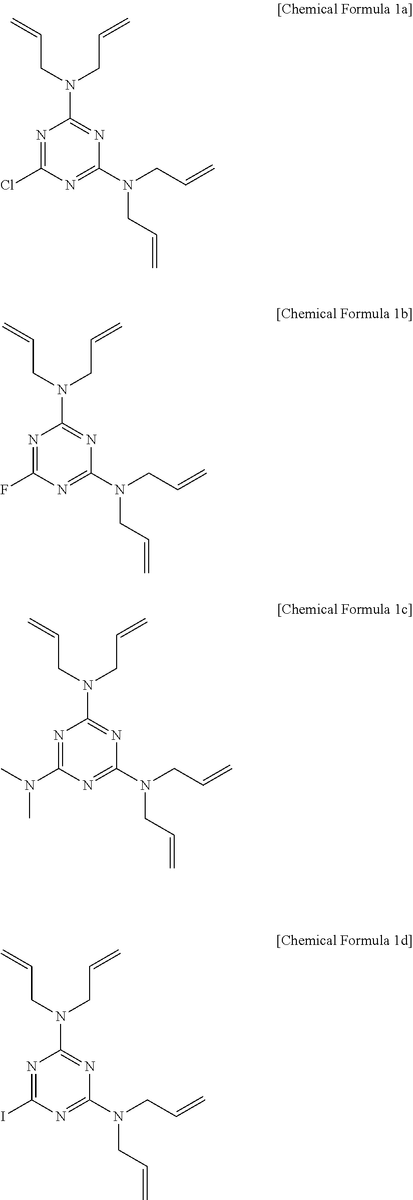

In some embodiments, the electrolyte additive may be the compound of Chemical Formula 1c.

According to another embodiment of the present invention, an electrolyte for a rechargeable lithium battery includes the additive.

The additive may be included in an amount of about 0.1 wt % to about 10 wt % based on the total weight of the electrolyte.

According to yet another embodiment of the present invention, a rechargeable lithium battery includes a positive electrode including a positive active material; a negative electrode including a negative active material; and an electrolyte, wherein the electrolyte includes a lithium salt, a non-aqueous organic solvent, and the additive.

The positive active material may be a lithium composite metal oxide having a 5V-grade spinel structure. In some embodiments, the positive active material may be Li₁₊ₓNi_{y}Mn_{2-y-z}M_{z}O_{4+w}, where M is selected from Al, Ti, Mg, Zn, or a combination thereof, 0≦x<0.2, 0.4≦y≦0.6, 0≦z≦0.2, and 0≦w≦0.1.

According to aspects of embodiments of the present invention, by forming a coating layer on the surface of a positive electrode, the electrolyte additive for a rechargeable lithium battery may prevent or reduce the elution of metal included in the positive active material at high temperatures. In addition, the electrolyte additive may decrease the deposition of metal ions on the surface of a negative electrode, thereby preventing or reducing the open-circuit voltage drop that is caused when a battery is allowed to stand at high temperatures for a long period of time. When applied to a rechargeable lithium battery including 5 V-grade spinel active material (which generally has insufficient storage characteristics at high temperatures), the storage characteristics at high temperatures are improved, and the battery has high power and high voltage.

### BRIEF DESCRIPTION OF THE DRAWINGS

FIG. 1 is a schematic view depicting a rechargeable lithium battery cell according to one embodiment.

FIG. 2 is a graph showing the results of observing the open-circuit voltage drop of battery cells according to Examples 1 to 3 and Comparative Examples 1 and 2 while being stored at high temperatures.

### DETAILED DESCRIPTION

Exemplary embodiments of this disclosure will hereinafter be described in detail. However, these embodiments are only exemplary, and this disclosure is not limited thereto.

As used herein, when a specific definition is not otherwise provided, the term “aliphatic saturated or unsaturated hydrocarbon group” may refer to a C1 to C30 aliphatic saturated or unsaturated hydrocarbon group, the term “aromatic hydrocarbon group” may refer to a C6 to C30 aromatic hydrocarbon group, and the term “heterocyclic group” may refer to either a C1 to C30 heteroaryl group or a C1 to C30 heterocycloalkyl group. As used herein, when a specific definition is not otherwise provided, the term “hetero” may refer to one including at least one atom selected from N, O, S, and P, instead of carbon.

As used herein, when a specific definition is not otherwise provided, the term “alkyl group” may refer to a C1 to C20 alkyl group, the term “alkenyl group” may refer to a C2 to C20 alkenyl group, the term “alkynyl group” may refer to a C2 to C20 alkynyl group, the term “cycloalkyl group” may refer to a C3 to C30 cycloalkyl group, and the term “aryl group” may refer to a C6 to C30 aryl group.

As used herein, when a definition is not otherwise provided, the term “combination thereof” refers to at least two substituents bound to each other by a single bond or a linker, or at least two substituents condensed to each other.

As used herein, when a definition is not otherwise provided, all the compounds or substituents may be substituted or unsubstituted. As used herein, when a definition is not otherwise provided, the term “substituted” may refer to at least one hydrogen atom substituted with at least one substituent selected from a halogen (F, Cl, Br, or I), a hydroxyl group, a nitro group, a cyano group, an imino group (═NH or ═NRₐ, Rₐ is a C1 to C10 alkyl group), an amino group (—NH₂, —NH(Rₐ), —N(R_{b})(R_{c}), wherein Rₐ to R_{c} are each independently a C1 to C10 alkyl group), an amidino group, a hydrazine or hydrazine group, a carboxyl group, a substituted or unsubstituted C1 to C10 alkyl group, a substituted or unsubstituted C6 to C30 aryl group, a substituted or unsubstituted C3 to C30 cycloalkyl group, a substituted or unsubstituted C3 to C30 heteroaryl group, or a substituted or unsubstituted C2 to C30 heterocycloalkyl group, instead of the at least one hydrogen of a functional group.

In the rechargeable lithium battery system using a 5 V-grade spinel active material based on spinel manganese, a significant drawback is the phenomenon that manganese (Mn) ions are eluted at high temperatures. Particularly, when the battery system including a 5 V-grade spinel-based positive active material is allowed to stand at high temperatures for a predetermined or set time, a LiPF₆ lithium salt is decomposed to generate HF according to the following reaction.

LiPF₆→LiF+PF₅

PF₅+H₂O→2HF+OPF₃

Due to the HF generated by the decomposition of lithium salt, Mn is eluted (generating Mn²⁺ ions) from the positive electrode, and the generated Mn²⁺ ions migrate to the negative electrode and reacted with electrons, thereby decomposing the ions on the surface of a negative electrode surface. When maintaining a battery at high temperatures, Mn is continuously eluted at the positive electrode, and Mn is continuously deposited at the negative electrode, thereby increasing the negative electrode potential to break the cell balance to cause capacity fading or reduction.

In order to reduce or prevent the elution of manganese ions at the positive electrode and the deposition of manganese ions on the surface of a negative electrode, and in order to improve the battery storage characteristics at a high temperature, a film is coated on the surface of a positive electrode using a triazine-based compound, having at least two amino groups substituted with hydrocarbon groups having a carbon-carbon double bond, as an electrolyte additive for a rechargeable lithium battery.

An electrolyte additive for a rechargeable lithium battery according to one embodiment of the present invention is a compound represented by the following Chemical Formula 1:

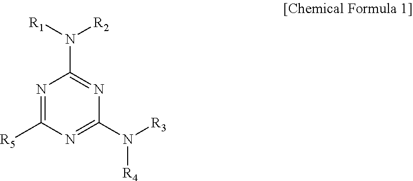

In Chemical Formula 1, R₁ to R₄ are each independently selected from hydrogen, an aliphatic saturated hydrocarbon group, an aliphatic unsaturated hydrocarbon group, an aromatic hydrocarbon group, a heterocyclic group, and/or a combination thereof, provided that at least one of R₁ or R₂ and at least one of R₃ or R₄ are each independently an unsaturated hydrocarbon group including at least one carbon-carbon double bond. R₅ is selected from hydrogen, a halogen, an aliphatic saturated or unsaturated hydrocarbon group, an aromatic hydrocarbon group, a hydroxyl group, a nitro group, a cyano group, an imino group, an amino group, an amidino group, a hydrazine group, a carboxyl group, a heterocyclic group, or a combination thereof.

In one embodiment, in Chemical Formula 1, R₁ to R₄ are each independently selected from a C1 to C20 alkyl group, a C2 to C20 alkenyl group, a C2 to C20 alkynyl group, an allyl group, a C3 to C30 cycloalkyl group, a C3 to C30 cycloalkenyl group, a C6 to C30 aryl group, or a C2 to C30 heterocyclic group, provided that at least one of R₁ or R₂ and at least one of R₃ or R₄ are each independently a C2 to C20 alkenyl group or an allyl group. R₅ is selected from hydrogen, a halogen, a C1 to C20 alkyl group, a C2 to C20 alkenyl group, a C2 to C20 alkynyl group, an allyl group, a C3 to C30 cycloalkyl group, a C3 to C30 cycloalkenyl group, a C6 to C30 aryl group, a C2 to C30 heterocyclic group, a hydroxyl group, a nitro group, a cyano group, an imino group, an amino group, an amidino group, a hydrazine group, a carboxyl group, or a combination thereof.

In another embodiment, in Chemical Formula 1, R₁ to R₄ are each independently a vinyl group or an allyl group, and R₅ is selected from a halogen, —NH₂, —NHR′, or —NR′R″, wherein R′ and R″ are each independently a C1 to C10 alkyl group.

In one embodiment, the electrolyte additive may be selected from compounds represented by the following Chemical Formulae 1a to 1d, or a mixture thereof:

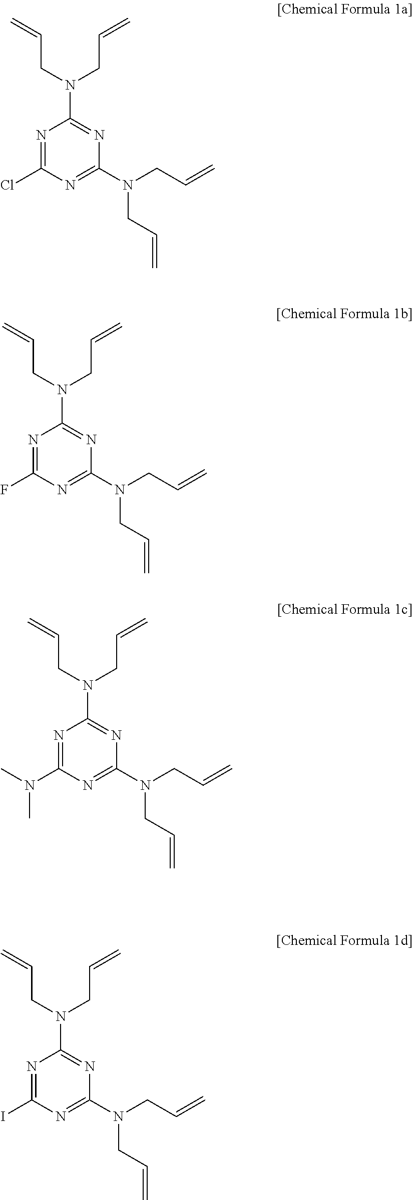

In another embodiment, the compound represented by the above Chemical Formula 1 may be a compound represented by the above Chemical Formula 1c, which includes nitrogen-containing substituents positioned in a tripodal shape.

Since the electrolyte additive has a low oxidation decomposition voltage and a high Highest Occupied Molecular Orbital (HOMO) as a result of the inclusion of at least two amino groups substituted with a hydrocarbon group having a carbon-carbon double bond, the additive relatively easily forms a coating layer on the positive electrode. As the result, when using the electrolyte additive in a rechargeable lithium battery, the elution of metal, for example, Mn included in the positive active material, and the deposition of Mn ions on the surface of the negative electrode are prevented or reduced, so as to prevent or reduce the open circuit voltage drop caused when the battery is allowed to stand at high temperatures. In addition, when the electrolyte additive is included in a rechargeable lithium battery, the battery may have a high voltage charge.

Particularly, when applied to an electric system using a spinel manganese-based a 5 V-grade spinel active material in which the performance may be seriously deteriorated at high temperatures, the additive is adsorbed on the surface of a positive electrode to capture the metal ions. Thereby, it may suppress the phenomenon where the eluted manganese ions are deposited at the negative electrode, so that the high temperature resistance may be remarkably improved.

Accordingly, a non-aqueous electrolyte for a rechargeable lithium battery including the electrolyte additive is provided.

The non-aqueous electrolyte for a rechargeable lithium battery according to one embodiment includes the compound represented by the above Chemical Formula 1 as an additive, a lithium salt, and a non-aqueous organic solvent.

Hereinafter, each component is described in detail.

Electrolyte Additive

The electrolyte additive is the same as described above.

The electrolyte additive may be included in an amount of about 0.1 wt % to about 10 wt % based on the total weight of the electrolyte. In some embodiments, when the electrolyte additive is included within the above range, a film on an electrode is easily formed, thereby providing improved storage characteristics at high temperatures. In one embodiment, the electrolyte additive may be included in an amount of about 0.1 wt % to about 2 wt % based on the total weight of the electrolyte, and in some embodiments, the electrolyte additive may be included in an amount of about 0.1 wt % to about 0.2 wt % based on the total weight of the electrolyte.

Lithium Salt

The lithium salt is dissolved in the non-aqueous organic solvent and supplies lithium ions in a rechargeable lithium battery, and improves lithium ion transfer between positive and negative electrodes. The lithium salt includes LiPF₆, LiBF₄, LiSbF₆, LiAsF₆, LiC₄F₉SO₃, LiClO₄, LiAlO₂, LiAlCl₄, LiN(CₓF₂ₓ₊₁SO₂)(C_{y}F_{2y+1}SO₂) (wherein, x and y are natural numbers), LiCl, LiI, LiB(C₂O₄)₂ (lithium bis(oxalato) borate, LiBOB), or a mixture thereof. The lithium salt is used as a supporting electrolytic salt.

The lithium salt may be used at a concentration of about 0.1 M to about 2.0 M. In some embodiments, when the lithium salt is included within the above concentration range, electrolyte performance and lithium ion mobility is improved due to optimal electrolyte conductivity and viscosity.

Non-Aqueous Organic Solvent

The non-aqueous organic solvent plays a role of transmitting ions taking part in the electrochemical reaction of a battery.

The non-aqueous organic solvent may include a carbonate-based, ester-based, ether-based, ketone-based, alcohol-based, or aprotic solvent.

The carbonate-based solvent may include dimethyl carbonate (DMC), diethyl carbonate (DEC), dipropyl carbonate (DPC), methylpropyl carbonate (MPC), ethylpropyl carbonate (EPC), methylethyl carbonate (MEC), ethylene carbonate (EC), propylene carbonate (PC), butylene carbonate (BC), and/or the like. The ester-based solvent may include methyl acetate, ethyl acetate, methylpropinonate, ethylpropinonate, γ-butyrolactone, decanolide, valerolactone, mevalonolactone, caprolactone, and/or the like. The ether-based solvent may include dibutyl ether, tetraglyme, diglyme, dimethoxyethane, 2-methyltetrahydrofuran, tetrahydrofuran, and/or the like. The ketone-based solvent may include cyclohexanone and/or the like. The alcohol-based solvent may include ethanol, isopropyl alcohol, and/or the like. The aprotic solvent include nitriles such as R—CN (wherein R is a C2 to C20 linear, branched, or cyclic hydrocarbon group, and may include a double bond, an aromatic ring, or an ether bond), amides such as dimethylformamide and/or dimethylacetamide, dioxolanes such as 1,3-dioxolane, sulfolanes, and/or the like.

The non-aqueous organic solvent may be used singularly or in a mixture. When the organic solvent is used in a mixture, its mixture ratio can be controlled in accordance with a desired performance of a battery.

The carbonate-based solvent may include a mixture of a cyclic carbonate and a linear carbonate. The cyclic carbonate and the linear carbonate may be mixed together in a volume ratio of about 1:1 to about 1:9, which may enhance performance of an electrolyte.

In addition, the non-aqueous organic solvent may be prepared by further adding an aromatic hydrocarbon-based solvent to the carbonate-based solvent. The carbonate-based solvent and the aromatic hydrocarbon-based solvent may be mixed together in a volume ratio of about 1:1 to about 30:1.

The aromatic hydrocarbon-based organic solvent may be an aromatic hydrocarbon-based compound represented by the following Chemical Formula 2:

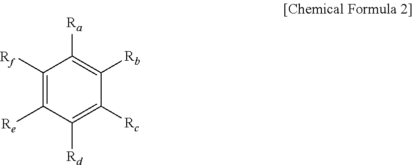

In Chemical Formula 2, Rₐ to R_{f} are each independently selected from hydrogen, a halogen, a C1 to C10 alkyl group, a C1 to C10 haloalkyl group, or a combination thereof.

The aromatic hydrocarbon-based organic solvent may be selected from benzene, fluorobenzene, 1,2-difluorobenzene, 1,3-difluorobenzene, 1,4-difluorobenzene, 1,2,3-trifluorobenzene, 1,2,4-trifluorobenzene, chlorobenzene, 1,2-dichlorobenzene, 1,3-dichlorobenzene, 1,4-dichlorobenzene, 1,2,3-trichlorobenzene, 1,2,4-trichlorobenzene, iodobenzene, 1,2-diiodobenzene, 1,3-diiodobenzene, 1,4-diiodobenzene, 1,2,3-triiodobenzene, 1,2,4-triiodobenzene, toluene, fluorotoluene, 1,2-difluorotoluene, 1,3-difluorotoluene, 1,4-difluorotoluene, 1,2,3-trifluorotoluene, 1,2,4-trifluorotoluene, chlorotoluene, 1,2-dichlorotoluene, 1,3-dichlorotoluene, 1,4-dichlorotoluene, 1,2,3-trichlorotoluene, 1,2,4-trichlorotoluene, iodotoluene, 1,2-diiodotoluene, 1,3-diiodotoluene, 1,4-diiodotoluene, 1,2,3-triiodotoluene, 1,2,4-triiodotoluene, xylene, or a mixture thereof.

The non-aqueous electrolyte may further include vinylene carbonate, an ethylene carbonate-based compound represented by the following Chemical Formula 3, or a combination thereof to improve cycle-life.

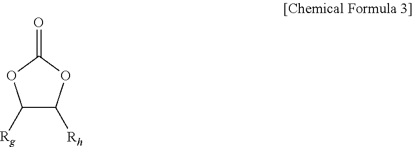

In Chemical Formula 3, R_{g} and Rₕ are each independently hydrogen, a halogen, a cyano group (CN), a nitro group (NO₂), or a C1 to C5 fluoroalkyl group, provided that at least one of R_{g} and Rₕ is a halogen, a cyano group (CN), a nitro group (NO₂), or a C1 to C5 fluoroalkyl group.

Examples of the ethylene carbonate-based compound include difluoro ethylenecarbonate, chloroethylene carbonate, dichloroethylene carbonate, bromoethylene carbonate, dibromoethylene carbonate, nitroethylene carbonate, cyanoethylene carbonate, fluoroethylene carbonate, and the like. The amount of the vinylene carbonate or the ethylene carbonate-based compound may be adjusted within an appropriate range to improve cycle life.

The non-aqueous electrolyte of the present invention may include the above components and thus may improve the high temperature characteristics of a rechargeable lithium battery.

According to another embodiment of the present invention, a rechargeable lithium battery including the non-aqueous electrolyte is provided.

The rechargeable lithium battery may be classified as a lithium ion battery, a lithium ion polymer battery, or a lithium polymer battery according to the presence of a separator and the kind of electrolyte used in the battery. The rechargeable lithium batteries may have a variety of shapes and sizes, and thus may be cylindrical, prismatic, or coin-type batteries and also, may be thin film batteries or rather bulky batteries in size. General structures and fabrication methods of the rechargeable lithium battery are known in the art.

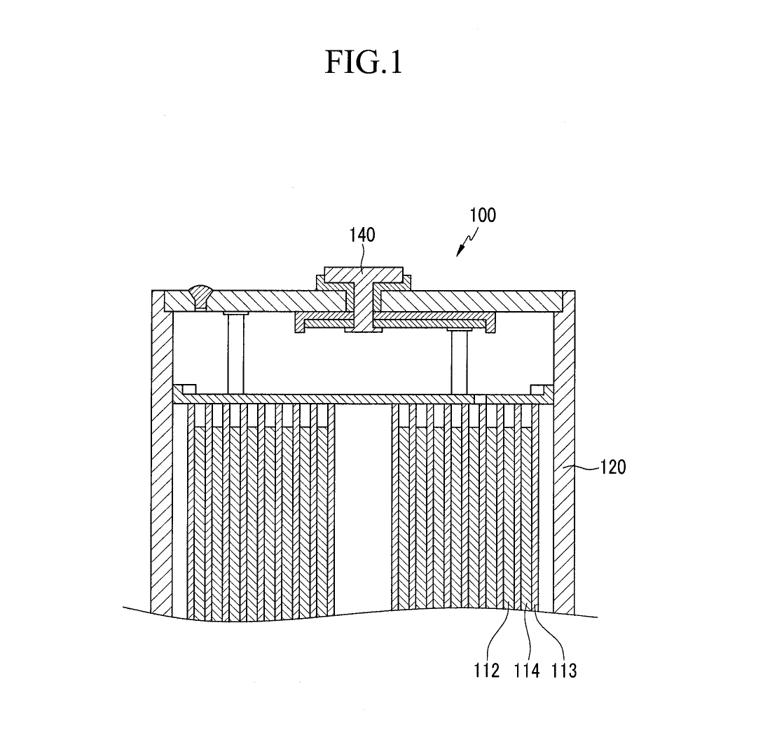

FIG. 1 is an exploded perspective view showing a rechargeable lithium battery in accordance with an embodiment. Referring to FIG. 1, the rechargeable lithium battery 100 is a cylindrical battery that includes a negative electrode 112, a positive electrode 114, a separator 113 disposed between the positive electrode 114 and the negative electrode 112, an electrolyte (not shown) impregnated in the negative electrode 112, the positive electrode 114, and the separator 113, a battery case 120, and a sealing member 140 sealing the battery case 120. The rechargeable lithium battery 100 is fabricated by sequentially stacking a negative electrode 112, a separator 113, and a positive electrode 114, and spiral-winding them and housing the wound product in the battery case 120.

The negative electrode 112 includes a current collector and a negative active material layer formed on the current collector, and the negative active material layer includes a negative active material.

The negative active material includes a material that reversibly intercalates/deintercalates lithium ions, a lithium metal, a lithium metal alloy, a material being capable of doping and dedoping lithium, or a transition metal oxide.

The material that reversibly intercalates/deintercalates lithium ions includes carbon materials. The carbon materials may be any generally-used carbon-based negative active material in a lithium ion rechargeable battery. Examples of the carbon material include crystalline carbon, amorphous carbon, and a combination thereof. The crystalline carbon may be non-shaped, or sheet, flake, spherical, or fiber shaped natural graphite or artificial graphite. The amorphous carbon may be a soft carbon, a hard carbon, a mesophase pitch carbonized product, fired coke, and/or the like.

Examples of the lithium metal alloy include lithium and a metal selected from Na, K, Rb, Cs, Fr, Be, Mg, Ca, Sr, Si, Sb, Pb, In, Zn, Ba, Ra, Ge, Al, or Sn.

Examples of the material being capable of doping and dedoping lithium include Si, SiOₓ (0<x<2), a Si—X₁ alloy (wherein X₁ is not Si and is an element selected from an alkali metal, an alkaline-earth metal, a Group 13 to 16 element, a transition element, a rare earth element, or a combination thereof), Sn, SnO₂, Sn—X₂ alloy (wherein X₂ is not Si and is an element selected from an alkali metal, an alkaline-earth metal, a Group 13 to 16 element, a transition element, a rare earth element, or combinations thereof), and the like. X₁ and X₂ may be an element selected from Mg, Ca, Sr, Ba, Ra, Sc, Y, Ti, Zr, Hf, Rf, V, Nb, Ta, Db, Cr, Mo, W, Sg, Tc, Re, Bh, Fe, Pb, Ru, Os, Hs, Rh, Ir, Pd, Pt, Cu, Ag, Au, Zn, Cd, B, Al, Ga, Sn, In, Ti, Ge, P, As, Sb, Bi, S, Se, Te, Po, or a combination thereof.

Examples of the transition metal oxide include vanadium oxide, lithium vanadium oxide, and the like.

The binder improves binding properties of the negative active material particles to one another and to a current collector. Examples of the binder include polyvinyl alcohol, carboxylmethyl cellulose, hydroxypropyl cellulose, polyvinylchloride, carboxylated polyvinylchloride, polyvinylfluoride, an ethylene oxide-containing polymer, polyvinylpyrrolidone, polyurethane, polytetrafluoroethylene, polyvinylidene fluoride, polyethylene, polypropylene, a styrene-butadiene rubber, an acrylated styrene-butadiene rubber, an epoxy resin, nylon, and the like, but the binder is not limited thereto.

The negative active material layer may optionally include a conductive material. The conductive material improves electrical conductivity of a negative electrode. Any electrically conductive material can be used as a conductive agent, unless it causes a chemical change. Examples of the conductive material include a carbon-based material such as natural graphite, artificial graphite, carbon black, acetylene black, ketjen black, a carbon fiber, and/or the like; a metal-based material of a metal powder or a metal fiber including copper, nickel, aluminum, silver, and/or the like; a conductive polymer such as a polyphenylene derivative; and a mixture thereof.

The current collector may be a copper foil, a nickel foil, a stainless steel foil, a titanium foil, a nickel foam, a copper foam, a polymer substrate coated with a conductive metal, or combinations thereof.

The positive electrode 114 includes a current collector and a positive active material layer disposed on the current collector.

The positive active material includes lithiated intercalation compounds that reversibly intercalate and deintercalate lithium ions. The positive active material may include a composite oxide including lithium and at least one selected from cobalt, manganese, and nickel. In particular, the following compounds may be used: LiₐA_{1-b}R_{b}D₂ (0.90≦a≦1.8 and 0≦b≦0.5); LiₐE_{1-b}R_{b}O_{2-c}D_{c} (0.90≦a≦1.8, 0≦b≦0.5, and 0≦c≦0.05); LiE_{2-b}R_{b}O_{4-c}D_{c} (0≦b≦0.5 and 0≦c≦0.05); LiₐNi_{1-b-c}Co_{b}R_{c}D_{α} (0.90≦a≦1.8, 0≦b≦0.5, 0≦c≦0.05, and 0<α≦2); LiₐNi_{1-b-c}Co_{b}R_{c}O_{2-α}Z_{α} (0.90≦a≦1.8, 0≦b≦0.5, 0≦c≦0.05, and 0<α<2); LiₐNi_{1-b-c}Co_{b}R_{c}O_{2-α}Z₂ (0.90≦a≦1.8, 0≦b≦0.5, 0≦c≦0.05, and 0<α<2); LiₐNi_{1-b-c}Mn_{b}R_{c}D_{α} (0.90≦a≦1.8, 0≦b≦0.5, 0≦c≦0.05, and 0<α≦2); LiₐNi_{1-b-c}Mn_{b}R_{c}O_{2-α}Z_{α} (0.90≦a≦1.8, 0≦b≦0.5, 0≦c≦0.05, and 0<α<2); LiₐNi_{1-b-c}Mn_{b}R_{c}O_{2-α}Z₂ (0.90≦a≦1.8, 0≦b≦0.5, 0≦c≦0.05, and 0<α<2); LiₐNi_{b}E_{c}G_{d}O₂ (0.90≦a≦1.8, 0≦b≦0.9, 0≦c≦0.5, and 0.001≦d≦0.1); LiₐNi_{b}CoₒMnₐGₑO₂ (0.90≦a≦1.8, 0≦b≦0.9, 0≦c≦0.5, 0≦d≦0.5, and 0.001≦e≦0.1); LiₐNiG_{b}O₂ (0.90≦a≦1.8 and 0.001≦b≦0.1); LiₐCoG_{b}O₂ (0.90≦a≦1.8 and 0.001≦b≦0.1); LiₐMnG_{b}O₂ (0.90≦a≦1.8 and 0.001≦b≦0.1); LiₐMn₂G_{b}O₄ (0.90≦a≦1.8 and 0.001≦b≦0.1); Li₁₊ₓNi_{y}Mn_{2-y-z}M_{z}O_{4+w} (where M is selected from Al, Ti, Mg, Zn, or a combination thereof, 0≦x<0.2, 0.4≦y≦0.6, 0≦z≦0.2, and 0≦w≦0.1), QO₂; QS₂; LiQS₂; V₂O₅; LiV₂O₅; LiTO₂; LiNiVO₄; Li_{(3-f)}/J₂(PO₄)₃ (0≦f≦2); Li_{(3-f)}Fe₂(PO₄)₃ (0≦f≦2); or LiFePO₄.

In the above chemical formulae, A is Ni, Co, Mn, or a combination thereof; R is Al, Ni, Co, Mn, Cr, Fe, Mg, Sr, V, a rare earth element, or a combination thereof; D is O, F, S, P, or a combination thereof; E is Co, Mn, or a combination thereof; Z is F, S, P, or a combination thereof; G is Al, Cr, Mn, Fe, Mg, La, Ce, Sr, V, or a combination thereof; Q is Ti, Mo, Mn, or a combination thereof; T is Cr, V, Fe, Sc, Y, or a combination thereof; and J is V, Cr, Mn, Co, Ni, Cu, or a combination thereof.

The compound may have a coating layer on its surface or may be mixed with a compound having a coating layer. The coating layer may include at least one coating element compound selected from an oxide of a coating element, a hydroxide of a coating element, an oxyhydroxide of a coating element, an oxycarbonate of a coating element, or a hydroxyl carbonate of a coating element. The compounds for a coating layer may be amorphous or crystalline. The coating element for a coating layer may include Mg, Al, Co, K, Na, Ca, Si, Ti, V, Sn, Ge, Ga, B, As, Zr, or a mixture thereof. The coating layer may be formed by a method having no negative influence on the properties of a positive active material by including these elements in the compound. For example, the method may include any coating method such as spray coating, dipping, or the like, but it is not illustrated in more detail, as it is known to those who work in the related fields.

Among them, the lithium composite metal oxide having a 5 V-class spinel structure capable of providing high power and high voltage, for example, Li₁₊ₓNi_{y}Mn_{2-y-z}M_{z}O_{4+w} (where M is selected from Al, Ti, Mg, Zn, or a combination thereof, 0≦x<0.2, 0.4≦y≦0.6, 0≦z≦0.2, and 0≦w≦0.1), is preferably used as an active material.

The positive active material layer may include a binder and a conductive material.

The binder improves binding properties of the positive active material particles to one another and to a current collector. Examples of the binder may include polyvinyl alcohol, carboxylmethyl cellulose, hydroxypropyl cellulose, diacetyl cellulose, polyvinylchloride, carboxylated polyvinylchloride, polyvinylfluoride, an ethylene oxide-containing polymer, polyvinylpyrrolidone, polyurethane, polytetrafluoroethylene, polyvinylidene fluoride, polyethylene, polypropylene, a styrene-butadiene rubber, an acrylated styrene-butadiene rubber, an epoxy resin, nylon, and/or the like, but the binder is not limited thereto.

The conductive material improves electrical conductivity of a negative electrode. Any electrically conductive material may be used as a conductive agent, unless it causes a chemical change. Examples of the conductive material include a carbon-based material such as natural graphite, artificial graphite, carbon black, acetylene black, ketjen black, a carbon fiber, and/or the like; a metal-based material of a metal powder or a metal fiber including copper, nickel, aluminum, silver, and/or the like; a conductive polymer such as a polyphenylene derivative; and a mixture thereof.

The current collector may be Al but it is not limited thereto.

Each of the negative electrode 112 and the positive electrode 114 may be manufactured by mixing the active material, a conductive material, and a binder to form an active material composition and coating the active material composition on a current collector. Electrode manufacturing methods are known to those skilled in the art, and thus, they are not described in detail in the present specification. The solvent may include N-methylpyrrolidone and the like, but it is not limited thereto.

The electrolyte is the same as described above.

According to the kind of rechargeable lithium battery, a separator 113 may be interposed between the positive electrode 114 and the negative electrode 112. The separator 113 may include polyethylene, polypropylene, polyvinylidene fluoride, or a multilayer structure including two or more layers, and it may also include a mixed multilayer such as a polyethylene/polypropylene 2-layered separator, a polyethylene/polypropylene/polyethylene 3-layered separator, a polypropylene/polyethylene/polypropylene 3-layered separator, or the like.

Hereinafter, the embodiments are illustrated in more detail with reference to examples. However, the following are exemplary embodiments and are not intended to be limiting.

### Example 1

A positive active material of LiNi_{0.5}Mn_{1.5}O₄ having a particle diameter of 10 μm, a carbon conductive material, and a polyvinylidene fluoride binder were mixed at a weight ratio of 94:3:3 and N-methyl-2-pyrrolidone was added thereto to provide a positive active material slurry. The obtained positive active material slurry was coated on a current collector of aluminum foil having a thickness of 20 μm and dried in a vacuum oven and compressed to provide a positive electrode.

A polyethylene separator was interposed between the obtained positive electrode and a negative electrode of graphite to provide an electrode assembly. The electrode assembly was compressed and inserted in a battery case for a 2016 coin cell. N²,N²,N⁴,N⁴-tetraallyl-6-iodo-1,3,5-triazine-2,4-diamine represented by the following Chemical Formula 1d was added to an electrolyte mixture including 1.15 M LiPF₆ dissolved in a mixed solvent of ethylene carbonate (EC)/ethylmethyl carbonate (EMC) (mixed at a volume ratio of 30:70). The compound of Chemical Formula 1d was included at 1 wt % based on the total weight of non-aqueous electrolyte. The non-aqueous electrolyte was injected into the electrode assembly and sealed to provide a 2016 coin cell.

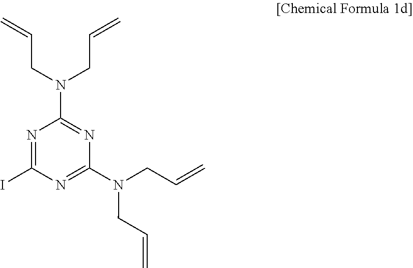 [SMILES: C=CCN(CC=C)c1nc(I)nc(N(CC=C)CC=C)n1]

### Example 2

A coin cell was fabricated in accordance with the same procedure as in Example 1, except that N²,N²,N⁴,N⁴-tetraallyl-6-fluoro-1,3,5-triazine-2,4-diamine represented by the following Chemical Formula 1b was used instead of the compound represented by Chemical Formula 1d.

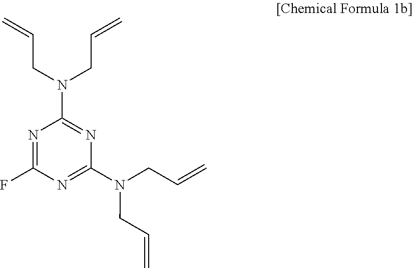 [SMILES: C=CCN(CC=C)c1nc(F)nc(N(CC=C)CC=C)n1]

### Example 3

A coin cell was fabricated in accordance with the same procedure as in Example 1, except that N²,N²,N⁴,N⁴-4-tetraallyl-N⁶,N⁶-dimethyl-1,3,5-triazine-2,4,6-triamine represented by the following Chemical Formula 1c was used instead of the compound represented by Chemical Formula 1d.

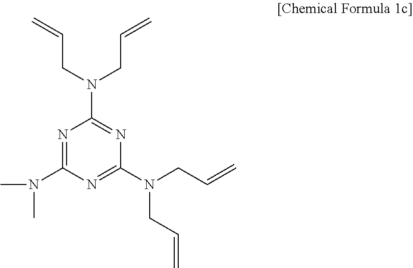 [SMILES: C=CCN(CC=C)c1nc(N(C)C)nc(N(CC=C)CC=C)n1]

### Comparative Example 1

A cell was fabricated in accordance with the same procedure as in Example 1, except that the additive was not included. That is, the electrolyte included 1.15 M LiPF₆ dissolved in a mixed solvent of ethylene carbonate (EC)/ethylmethyl carbonate (EMC) (mixed at a volume ratio of 30:70) to provide a non-aqueous electrolyte.

### Comparative Example 2

A cell was fabricated in accordance with the same procedure as in Example 1, except that 2,4,6-trichloro-1,3,5-triazine (Chemical Formula 4) was used instead of the compound represented by Chemical Formula 1d.

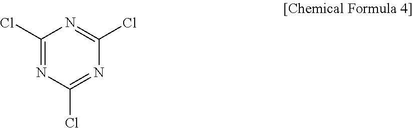

### Experimental Example

A charge and discharge test was performed on the cells obtained in Examples 1 to 3 and Comparative Examples 1 and 2.

First, a charge/discharge test was performed one time at the standard charge/discharge current density of 0.2 C/0.2 C, an end of charge voltage of 4.8 V (Li/graphite), and the end of discharge voltage of 3.0 V (Li/graphite). Then, after performing the battery charge/discharge for 2 times at 0.1 C/0.1 C, the battery was charged at a charge current of 0.5 C for once and the charged battery was stored in a thermostat chamber at a high temperature (i.e., 60° C.) for 50 days.

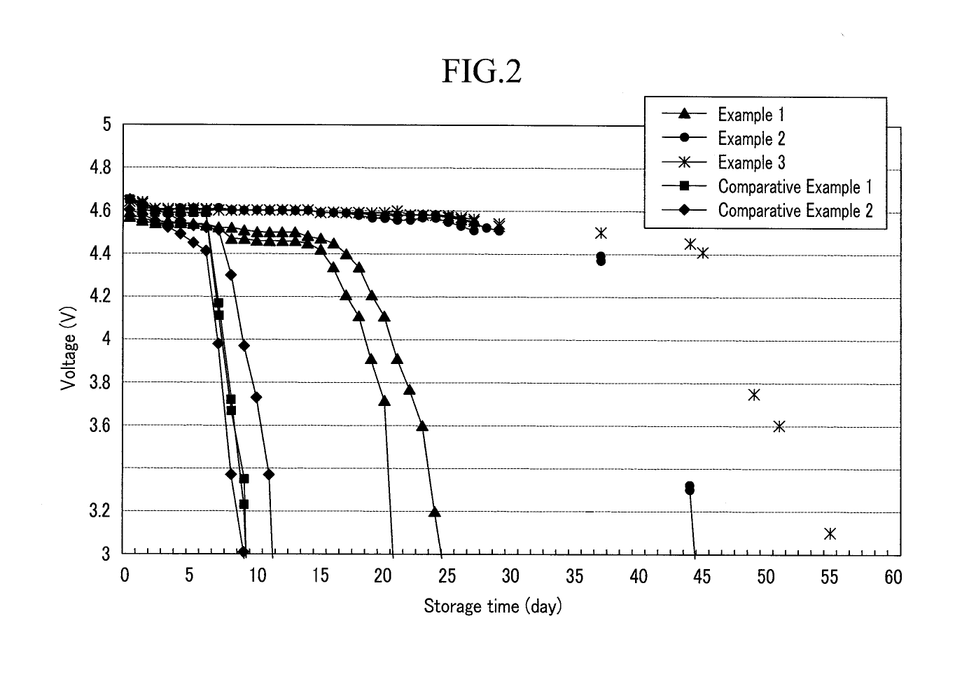

The initial discharge capacity and efficiency were measured, and the results are shown in the following Table 1. The measurement value is averaged. The open circuit voltage drop was observed during storing at a high temperature (i.e., 60° C.), and the results are shown in FIG. 2.

TABLE 1

| | Initial discharge capacity | Initial discharge capacity |
|---|---|---|
| | (mAh/g) | efficiency (%) |
| Comparative | 121.6 | 87.8 |
| Example 1 | | |
| Comparative | 99.2 | 72.5 |
| Example 2 | | |
| Example1 | 90.9 | 79.3 |
| Example2 | 119.2 | 82.1 |
| Example3 | 116.5 | 80.1 |

As shown in Table 1 and FIG. 2, the cells of Examples 1 to 3 using the triazine derivative additive having a structure of Chemical Formula 1 had lower initial discharge capacity than the battery cell of Comparative Example 1 including no electrolyte additive. However, when storing the battery cells having 5 V-grade spinel active material at a high temperature (e.g., 60° C.), the open circuit voltage (OCV) of Comparative Example 1, including no electrolyte additive, dropped for 10 days before having an OCV of 3 V. However, Examples 1 to 3 including a triazine derivative additive according to embodiments of the present invention had OCVs that dropped for 25 days, 45 days, and 53 days, respectively, before having an OCV of 3 V. From these results, it is understood that the storage characteristics of batteries at high temperatures are improved by using the triazine derivative additive according to embodiments of the invention.

The battery cells of Examples 1 to 3 using the triazine derivative additive of Chemical Formula 1 had improved initial discharge capacity efficiency compared to the battery cell of Comparative Example 2 including the electrolyte additive of the triazine-based compound having a different triazine additive including a different substituent.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims and equivalents thereof.

What is claimed is:

1. An electrolyte additive for a rechargeable lithium battery represented by the following Chemical Formula 1: 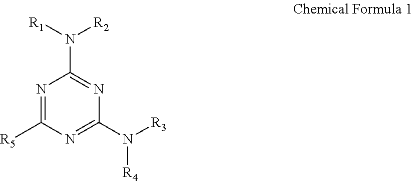
wherein,
R₁ to R₄ are each independently selected from the group consisting of hydrogen, an aliphatic saturated hydrocarbon group, an aliphatic unsaturated hydrocarbon group, an aromatic hydrocarbon group, a heterocyclic group, and a combination thereof, provided that at least one of R₁ or R₂ and at least one of R₃ or R₄ are independently an unsaturated hydrocarbon group including at least one carbon-carbon double bond, and
R₅ is selected from the group consisting of hydrogen, a halogen, an aliphatic saturated or unsaturated hydrocarbon group, an aromatic hydrocarbon group, a hydroxyl group, a nitro group, a cyano group, an imino group, an amino group, an amidino group, a hydrazine group, a carboxyl group, a heterocyclic group, and a combination thereof.

2. The electrolyte additive for a rechargeable lithium battery of claim 1, wherein:
R₁ to R₄ are each independently selected from the group consisting of a C1 to C20 alkyl group, a C2 to C20 alkenyl group, a C2 to C20 alkynyl group, an allyl group, a C3 to C30 cycloalkyl group, a C3 to C30 cycloalkenyl group, a C6 to C30 aryl group, and a C2 to C30 heterocyclic group, provided that at least one of R₁ or R₂ and at least one of R₃ or R₄ are independently a C2 to C20 alkenyl group or an allyl group, and
R₅ is selected from the group consisting of hydrogen, a halogen, a C1 to C20 alkyl group, a C2 to C20 alkenyl group, a C2 to C20 alkynyl group, an allyl group, a C3 to C30 cycloalkyl group, a C3 to C30 cycloalkenyl group, a C6 to C30 aryl group, a C2 to C30 heterocyclic group, a hydroxyl group, a nitro group, a cyano group, an imino group, an amino group, an amidino group, a hydrazine group, a carboxyl group, and a combination thereof.

3. The electrolyte additive for a rechargeable lithium battery of claim 1, wherein:
R₁ to R₄ are each independently a vinyl group or an allyl group, and
R₅ is selected from the group consisting of a halogen, —NH₂, —NHR′, and —NR′R″, wherein R′ and R″ are each independently a C1 to C10 alkyl group.

4. The electrolyte additive for a rechargeable lithium battery of claim 1, wherein the electrolyte additive is selected from the group consisting of compounds represented by the following Chemical Formula 1a to 1d and a mixture thereof:

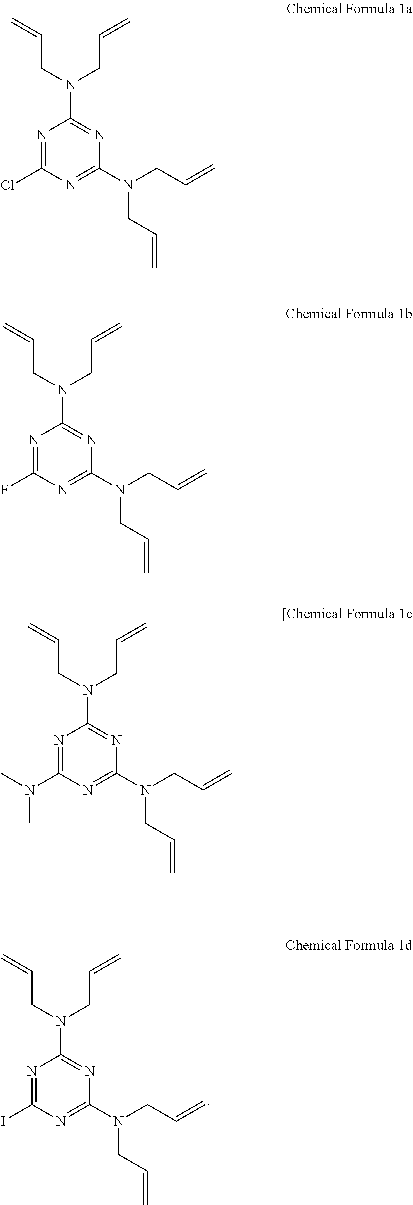

5. The electrolyte additive for a rechargeable lithium battery of claim 4, wherein the electrolyte additive is a compound represented by Chemical Formula 1c.

6. An electrolyte for a rechargeable lithium battery comprising:
a lithium salt;
a non-aqueous organic solvent; and
an electrolyte additive represented by the following Chemical Formula 1: 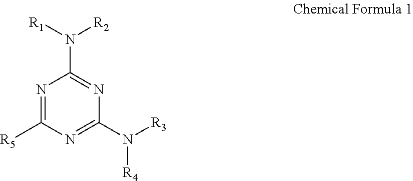
wherein,
R₁ to R₄ are each independently selected from the group consisting of hydrogen, an aliphatic saturated hydrocarbon group, an aliphatic unsaturated hydrocarbon group, an aromatic hydrocarbon group, a heterocyclic group, and a combination thereof, provided that at least one of R₁ or R₂ and at least one of R₃ or R₄ are independently an unsaturated hydrocarbon group including at least one carbon-carbon double bond, and
R₅ is selected from the group consisting of hydrogen, a halogen, an aliphatic saturated or unsaturated hydrocarbon group, an aromatic hydrocarbon group, a hydroxyl group, a nitro group, a cyano group, an imino group, an amino group, an amidino group, a hydrazine group, a carboxyl group, a heterocyclic group, and a combination thereof.

7. The electrolyte of claim 6, wherein the electrolyte additive is selected from the group consisting of compounds represented by the following Chemical Formulae 1a to 1d and a mixture thereof:

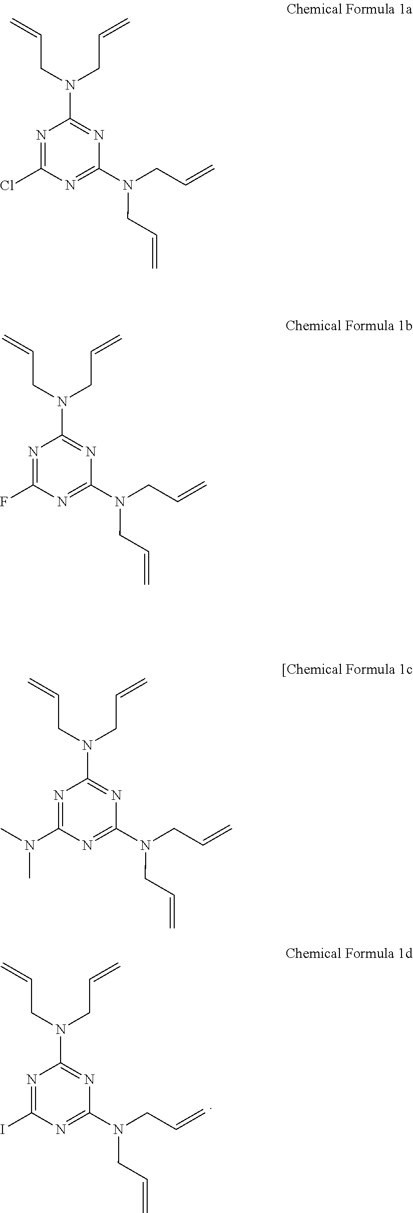

8. The electrolyte of claim 6, wherein the electrolyte additive is included in a range of about 0.1 wt % to about 10 wt % based on the total weight of electrolyte.

9. A rechargeable lithium battery comprising:
a positive electrode comprising a positive active material;
a negative electrode comprising a negative active material; and
an electrolyte comprising a lithium salt, a non-aqueous organic solvent, and an electrolyte additive represented by the following Chemical Formula 1: 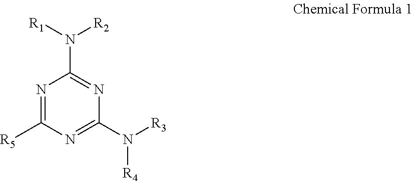
wherein,
R₁ to R₄ are each independently selected from the group consisting of hydrogen, an aliphatic saturated hydrocarbon group, an aliphatic unsaturated hydrocarbon group, an aromatic hydrocarbon group, a heterocyclic group, and a combination thereof, provided that at least one of R₁ or R₂ and at least one of R₃ or R₄ are independently an unsaturated hydrocarbon group including at least one carbon-carbon double bond, and
R₅ is selected from the group consisting of hydrogen, a halogen, an aliphatic saturated or unsaturated hydrocarbon group, an aromatic hydrocarbon group, a hydroxyl group, a nitro group, a cyano group, an imino group, an amino group, an amidino group, a hydrazine group, a carboxyl group, a heterocyclic group, and a combination thereof.

10. The rechargeable lithium battery of claim 9, wherein the positive active material has a 5 V-class spinet structure.

11. The rechargeable lithium battery of claim 9, wherein the positive active material is Li₁₊ₓNi_{y}Mn_{2-y-z}M_{z}O_{4+w}, wherein M is selected from the group consisting of Al, Ti, Mg, Zn, and a combination thereof, 0≦x<0.2, 0.4≦y≦0.6, 0≦z≦0.2, and 0≦w≦0:1.

12. The rechargeable lithium battery of claim 9, wherein electrolyte additive is selected from the group consisting of compounds represented by the following Chemical Formula 1a to 1d and a mixture thereof:

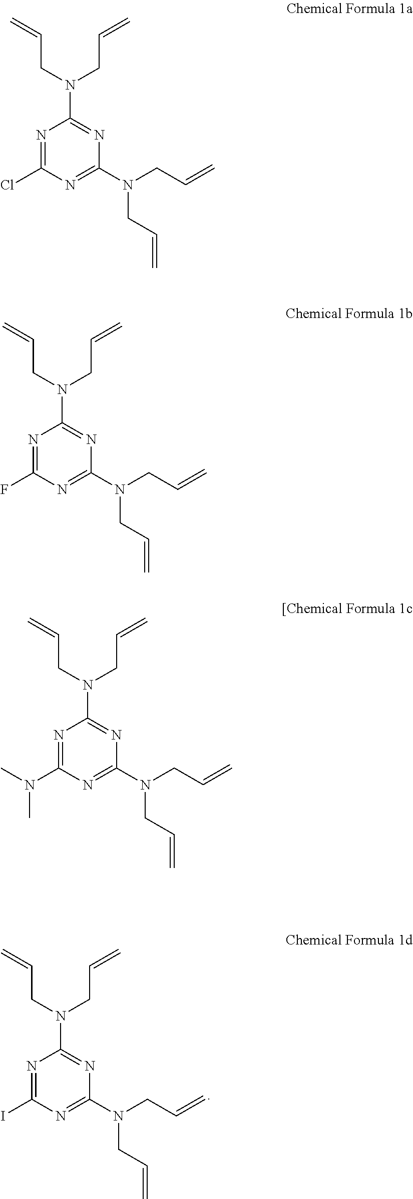

13. The rechargeable lithium battery of claim 9, wherein the electrolyte additive is included in a range of about 0.1 wt % to about 10 wt % based on the total weight of electrolyte.
